(19) United States
(12) Patent Application Publication
(10) Pub. No.: US 2020/0043587 A1
(43) Pub. Date: Feb. 6, 2020

(54) Method for Monitoring and Dispensing Controlled Substances

(71) Applicant: David Seltzer, Granite City, IL (US)

(72) Inventor: David Seltzer, Granite City, IL (US)

(21) Appl. No.: 16/056,099

(22) Filed: Aug. 6, 2018

(51) Int. Cl. G16H 20/13; G16H 15/00; G16H 10/60

(52) U.S. Cl. CPC G16H 20/13; G16H 10/60; G16H 15/00

(57) ABSTRACT

A method for monitoring and dispensing controlled substances is provided with a plurality of medical records stored on an external server. Each of the medical records consists of at least one controlled substance data that details the medication required by a patient. When a specific medical record is selected from the plurality of medical records, the specific medical record is matched with a controlled substance stored in a substance storage unit. Based on the at least one controlled substance data, a specified volume of the controlled substance is dispensed. Upon dispensing the specified volume into a syringe or other comparable holding device, the specified volume in the syringe is labelled with patient identification information. As a result, misuse and waste is prevented.

### FIELD OF THE INVENTION

The present invention generally relates to a method of monitoring and dispensing controlled substances. More specifically, the present invention controls excess distribution of controlled substances and diversion of the substances to unauthorized users.

### BACKGROUND OF THE INVENTION

Opioid overdose has been a major concern in the recent past. Misuse of and addiction to opioids has had a significant impact on not only public health, but also on social and economic welfare. As a solution to the conflict, the government has significantly reduced overall opioid production in the recent past. Thus, monitoring the distribution of the available opioids is vital to ensure that all patients are supplied with the required medication.

With current opioid distribution systems, the medical staff issuing the controlled substances must complete the related documentation process as well. Therefore, in addition to caring for the patient, the medical staff also needs to document the dosages given to each patient, update the medical information for each patient etc. By having to do the documentation process during the time allocated for the patient, the overall time the medical professional can devote to the patient is significantly reduced.

Another issue with existing distribution systems is excess distribution. Most of the time the prefilled manufacture amounts are greater than the prescribed amount, and the difference in quantities has resulted in excess distribution. With the limited availability of opioids, the excess distribution can lead to some patients not getting the required medication when needed. The excess distribution can also lead to the opioids being wasted or misused. Therefore, a method for monitoring the distribution of controlled substances is clearly required.

The objective of the present invention is to address the aforementioned issues. In particular, the present invention introduces a method to monitor the distribution and the use of controlled substances such that the waste and abuse of controlled substances is eliminated. Additionally, the present invention ensures that only the prescribed amounts of a certain medication is given to a patient.

### BRIEF DESCRIPTION OF THE DRAWINGS

FIG. 1A is a flowchart illustrating the basic overall process of the present invention, and

FIG. 1B is a continuation thereof, further illustrating the basic overall process of the present invention.

FIG. 2 is a flowchart illustrating the basic overall process of refilling the substance storage unit.

FIG. 3 is a flowchart illustrating the basic overall process of measuring the new level of the specific controlled substance by weight.

FIG. 4 is a flowchart illustrating the basic overall process of measuring the new level of the specific controlled substance by volume.

FIG. 5 is a flowchart illustrating the basic overall process of retrieving the volume of the specific controlled substance.

FIG. 6 is a flowchart illustrating the basic overall process of transferring the patient identification and updating the dosage information for the specific medical record.

FIG. 7 is a flowchart illustrating the basic overall process of using a plurality of pipettes in the dispensing unit.

FIG. 8 is a flowchart illustrating the basic overall process of using a plurality of syringes in the labelling unit.

### DETAIL DESCRIPTIONS OF THE INVENTION

All illustrations of the drawings are for the purpose of describing selected versions of the present invention and are not intended to limit the scope of the present invention.

The present invention introduces a method that can monitor the use and the distribution of controlled substances. Preferably, the present invention is intended to be used with intravenous schedule II drugs or other comparable controlled substances. Since the monitoring and the distribution process introduced by the present invention is automated, the need to manually document controlled substance related data is eliminated. Additionally, since the present invention is designed to dispense an exact amount of a controlled substance, the overall waste of the controlled substance is also eliminated. Since an excess of the controlled substance is not released to the patient, the overall possibility of misusing the controlled substance is eliminated.

To execute the intended functionalities, the present invention is provided with a plurality of medical records which is managed by an external server. A specific controlled substance a patient requires is monitored and distributed according to the information stored on each of the plurality of medical records. To provide the specific controlled substance as required, the present invention is also provided with a substance storage unit which stores a plurality of controlled substances. Each of the plurality of controlled substances is provided with a current quantity volume that records the initial volume of each of the plurality of controlled substances. The preferred embodiment of the present invention is intended to be used with intravenous schedule II drugs. Therefore, the plurality of controlled substances stored in the substance storage unit can be selected from a group comprising, but not limited to, Hydromorphone, Methadone, Meperidine, Oxycodone, Morphine, Fentanyl, and Codeine. However, the present invention can also be used with other controlled substances as well. The size and shape of the substance storage unit can vary. Preferably, the substance storage unit will be a secure metal container that comprises a set of sub-compartments, wherein the set of sub-compartments is used to hold each of the plurality of controlled substances. As an example, if three controlled substances are held within the substance storage unit, the three controlled substances will be held in three separate sub-compartments within the substance storage unit.

When the present invention is in use, a selection is received from a computing device, wherein the selection corresponds to a specific medical record selected from the plurality of medical records. Since the plurality of medical records is used to access the substance storage unit, each of the plurality of medical records comprises at least one controlled substance data. To select the specific medical record, the present invention is provided with an authorized user account which is managed by the external server. Preferably, the authorized user account will be operated by a medical professional. The limited access to the plurality of medical records and the substance storage unit is essential for safety purposes. Moreover, since the plurality of medical records is stored on the external server, the need to manually update each of the plurality of medical records is eliminated.

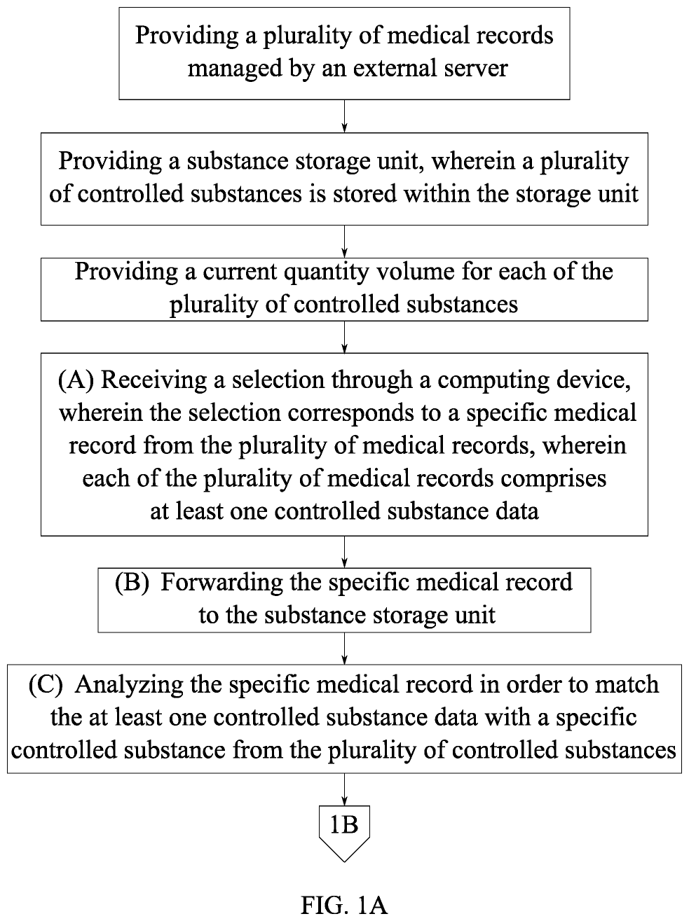

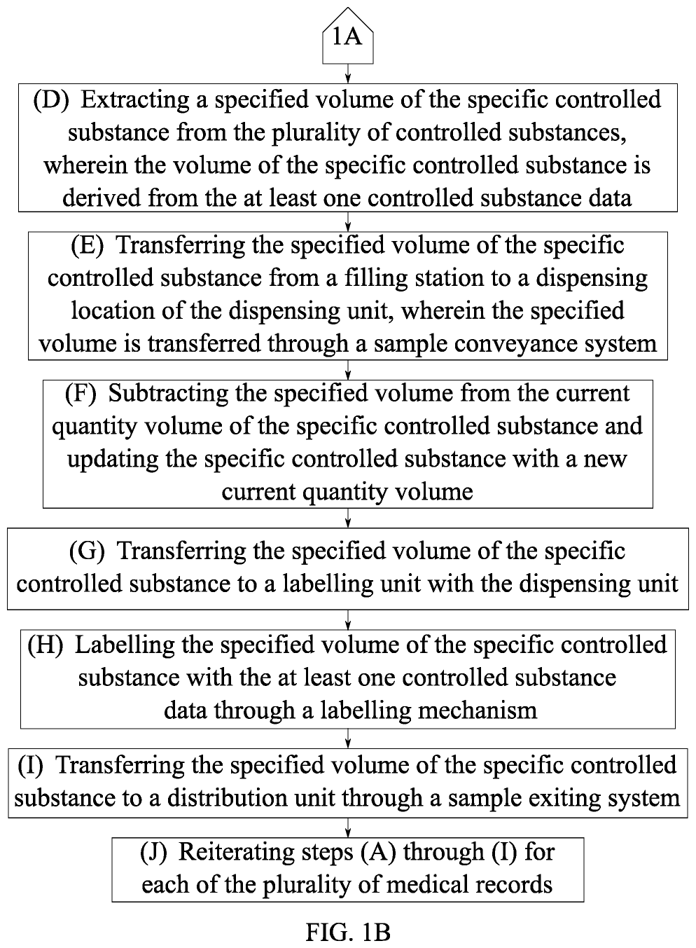

As illustrated in FIG. 1A, when the selection for the specific medical record is received through a computing device, the specific medical record is forwarded to the substance storage unit so that the substance storage unit can initiate the controlled substance monitoring and distribution process. The present invention analyzes the specific medical record in order to match the at least one controlled substance data with a specific controlled substance from the plurality of controlled substances. As an example, if the at least one controlled substance data requests Morphine, the present invention matches the specific medical record with the sub-compartment containing Morphine, wherein the sub-compartment is within the substance storage unit. Upon matching the at least one controlled substance data with the specific controlled substance, the present invention extracts a specified volume of the specific controlled substance from the plurality of controlled substances, wherein the specified volume of the specific controlled substance is determined by the at least one controlled substance data. In reference to the previous example, if 5-milliliters (ml) of Morphine is required in the at least one controlled substance data of the specific medical record, 5-ml of Morphine is extracted from the sub-compartment storing Morphine. Since the exact amount of the specific controlled substance is extracted, the specific controlled substance is not wasted.

As illustrated in FIG. 1B, upon extracting the specified volume of the specific controlled substance, the present invention transfers the specified volume of the specific controlled substance from a filling station to a dispensing location of a dispensing unit, which dispenses the specified volume of the specific controlled substance to be sent to the patient. The specified volume is transferred through a sample conveyance system which can be, but is not limited to, a conveyor belt. Preferably, the specified volume of the specific controlled substance will be dispensed to a syringe or other comparable holding device. When the specified volume of the specific controlled substance is extracted and dispensed, the specified volume is subtracted from the current quantity volume of the specific controlled substance. Subsequently, the present invention updates the specific controlled substance with a new current quantity volume. Referring to the previous example, if the sub-compartment with Morphine initially held 100-ml, after extracting 5-ml, the new current quantity volume of the specific controlled substance is updated to be 95-ml.

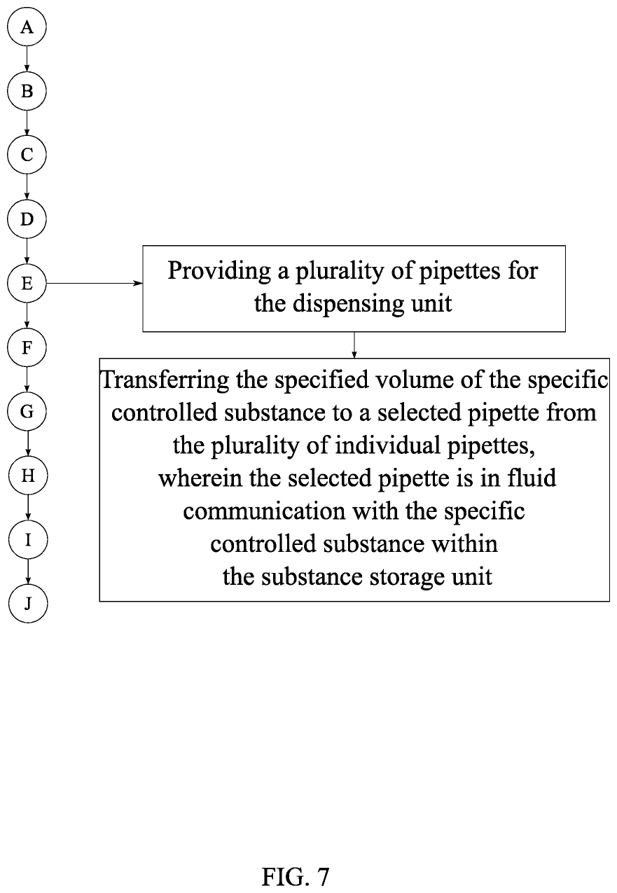

To accurately dispense the specified volume of the specific controlled substance, the dispensing unit is provided with a plurality of pipettes. As shown in FIG. 7, each of the plurality of pipettes is in fluid communication with a corresponding controlled substance from the plurality of controlled substances. For instance, if three controlled substances are stored in the substance storage unit, the plurality of pipettes will consist of three pipettes. Therefore, referring to the previous example, a selected pipette from the plurality of pipettes will be in fluid communication with the sub-compartment storing Morphine. As a result, the present invention can transfer the specified volume of the specific controlled substance to the selected pipette.

When the specified volume of the specific controlled substance is dispensed as required, the present invention transfers the specified volume of the specific controlled substance to a labelling unit through the dispensing unit. At the labelling unit, the specified volume of the specific controlled unit is labelled with the at least one controlled substance data through a labelling mechanism. The specified volume of the specific controlled substance will be labelled with information which can be, but is not limited to, medication name and medication quantity. Referring to the previous example, the specified volume of the specific controlled substance will be labelled as 5-ml of Morphine.

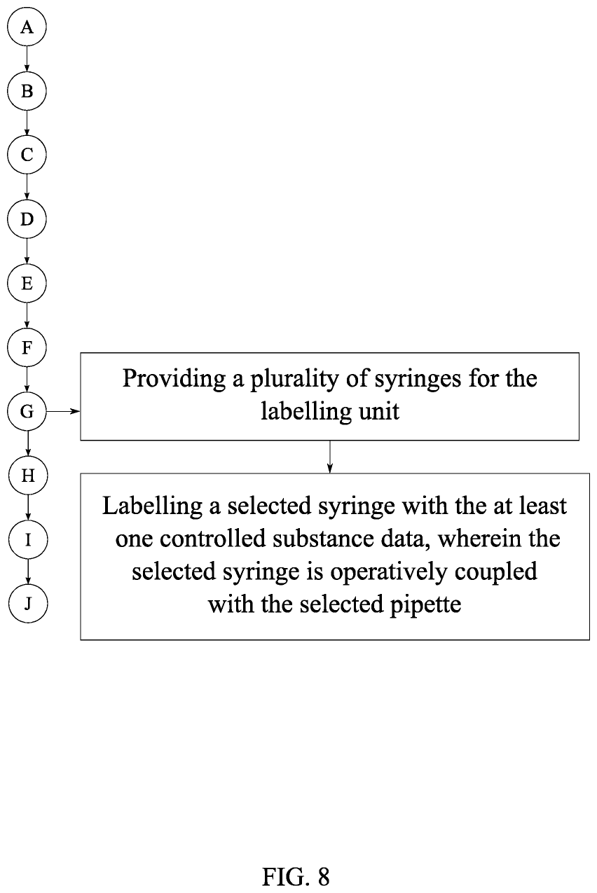

In the preferred embodiment of the present invention, the labelling unit is provided with a plurality of syringes. As shown in FIG. 8, a selected syringe of the plurality of syringes is operatively coupled with a selected pipette from the plurality of pipettes. As a result, when the present invention transfers the specified volume of the specific controlled substance to the labelling unit, the selected syringe is positioned adjacent the selected pipette. Therefore, with reference to the previous example, the selected syringe will be labelled as 5-ml Morphine. A mechanical arm or other comparable means can be used to place the selected syringe adjacent the selected pipette.

When the labelling process is complete, a sample exiting system of the present invention transfers the specified volume of the specific controlled substance to a distribution unit which allows the medical professional to retrieve the specified volume of the specific controlled substance. The medical professional is granted access to the distribution unit through the authorized user account. The process is repeated for each of the plurality of medical records. Since only prescribed amounts are distributed, the plurality of controlled substances is not wasted. Additionally, by having limited access, the overall misuse of the plurality of controlled substances is prevented. Preferably, the distribution unit will consist of a sliding door or other comparable means that requires authentication prior to opening.

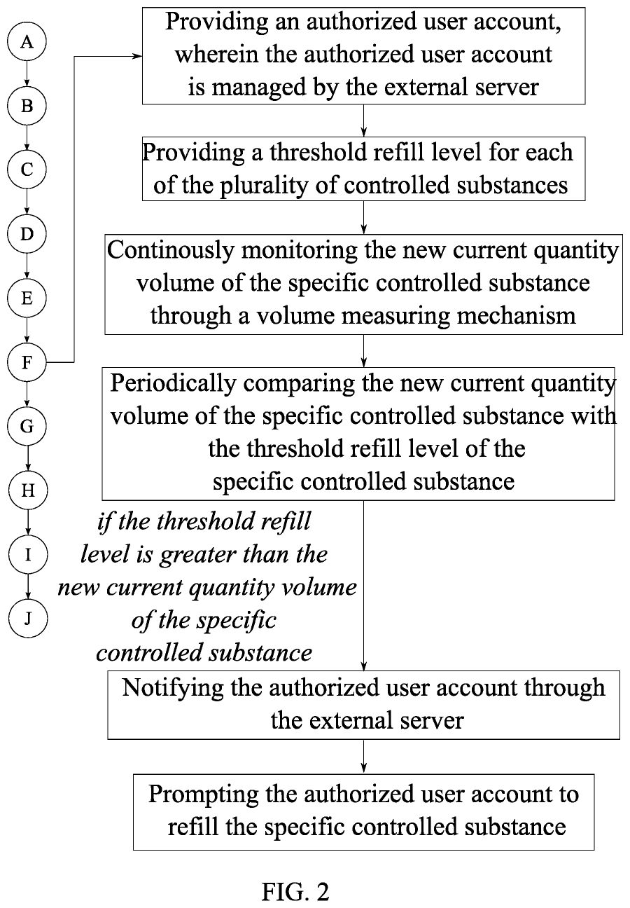

As discussed earlier, when the specified volume of the specific controlled substances is dispensed, the plurality of controlled substances is updated with a new current quantity volume of the specific controlled substance. Constantly updating and monitoring the new level of the specific controlled substance is important for refilling purposes. By doing so, the present invention ensures that the specific controlled substance is always available for a patient. Continuously monitoring the new current quantity volume of the specific controlled substance through a volume measuring mechanism is also important to ensure that an excess amount of the specific controlled substance is not extracted through the present invention. As illustrated in FIG. 2, to refill the specific controlled substance or any other arbitrary controlled substance of the plurality of controlled substances, the present invention is provided with a threshold refill level for each of the plurality of controlled substances. While monitoring the new current quantity volume of the specific controlled substance, the present invention periodically compares the new current quantity volume of the specific controlled substance with the threshold refill level of the specific controlled substance. The time interval for comparing can be predetermined by the authorized user account. As an example, if the time interval is 24-hours, the present invention compares the new current quantity volume of the specific controlled substance with the threshold refill level of the specific controlled substance every 24-hours.

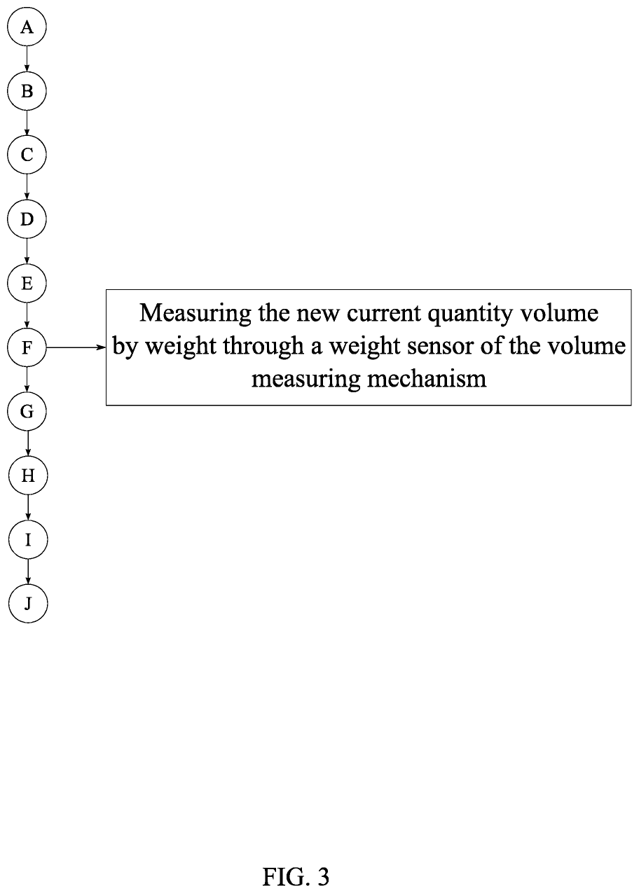

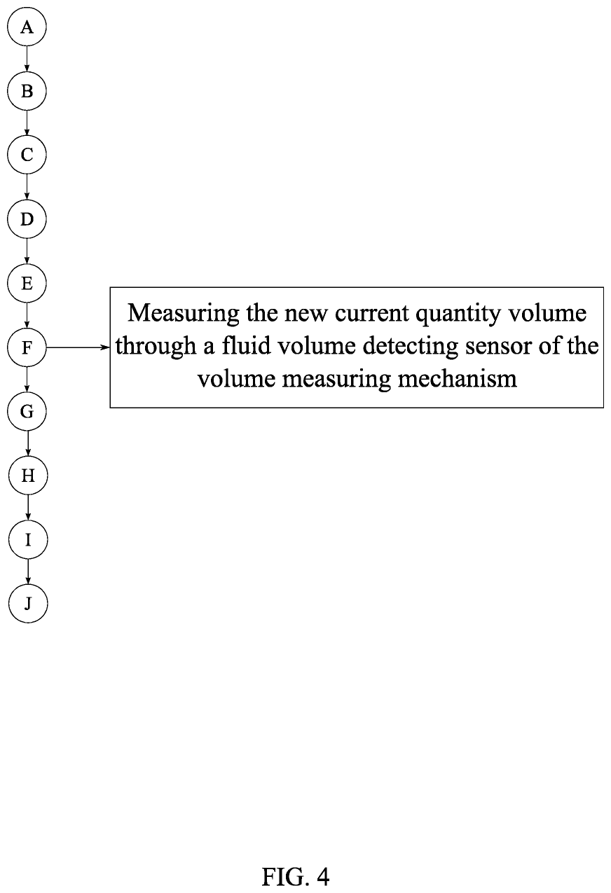

If the threshold refill level is greater than the new current quantity volume of the specific controlled substance, the present invention notifies the authorized user account through the external server. Next, the present invention prompts the authorized user account to refill the specific controlled substance so that the specific controlled substance is available upon request. More specifically, the authorized user account is prompted to refill the specific controlled substance such that the new current quantity volume is greater than the threshold refill level. Referring to the previous example wherein the specific controlled substance was Morphine, if the new level of the specific controlled substance is 45-ml, and the threshold refill level for Morphine is 50-ml, the authorized user account is notified through the external server. As illustrated in FIG. 3 and FIG. 4, the new current quantity volume of the specific controlled substance and the threshold refill level for each of the plurality of controlled substances can be measured by volume or by weight. Sensors and other measuring mechanisms can be used to measure and monitor the new current quantity volume of the specific controlled substance and the threshold refill level of the plurality of controlled substances. More specifically, a weight sensor or a fluid volume detecting sensor of the volume measuring mechanism can be used.

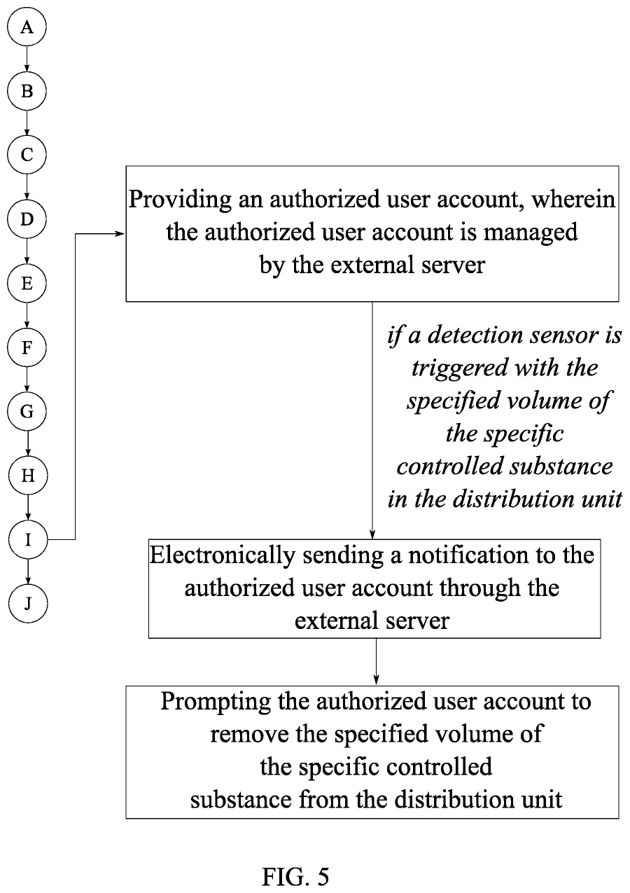

As described earlier, only the authorized user account can access the substance storage unit for refilling purposes. Similarly, for safety purposes, only the authorized user account can access the distribution unit to retrieve the specified volume of the specific controlled substance. As illustrated in FIG. 5, to retrieve the specified volume of the specific controlled substance from the distribution unit, the present invention electronically sends a notification to the authorized user account if a detection sensor is triggered with the specified volume of the specific controlled substance in the distribution unit. After notifying the authorized user account through the external server, the authorized user account is prompted to remove the specified volume of the specific controlled substance from the distribution unit. The authorized user account can be granted access through a face recognition application, a fingerprint identification method, or other comparable authentication method.

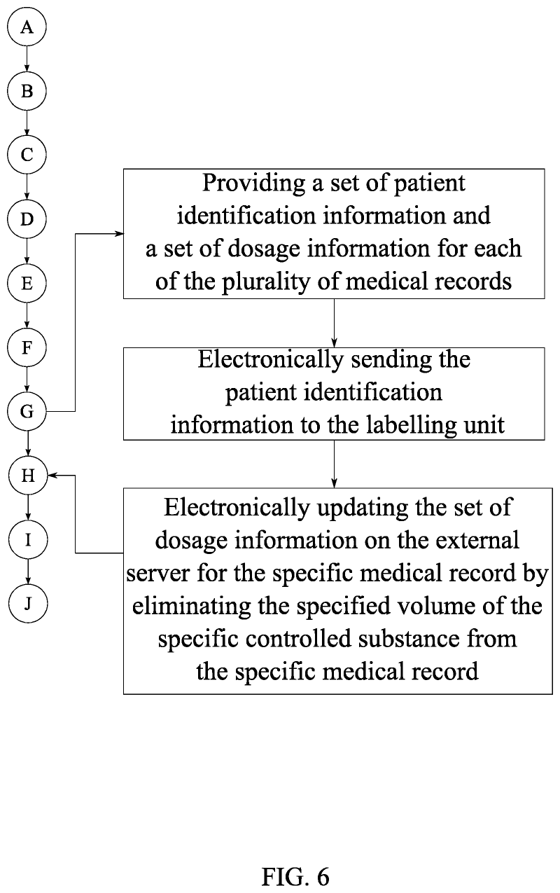

As described earlier, each of the plurality of medical records comprises at least one controlled substance data. Referring to the previous example, the specific medical record contains information regarding Morphine. As shown in FIG. 6, in addition to the at least one controlled substance data, a set of patient identification information and a set of dosage information is provided for each of the plurality of medical records. The set of patient identification information can be, but is not limited to, patient name, age, and address. On the other hand, the set of dosage information can contain information such as medication times and medication dosages. To ensure that the specified volume of the specific controlled substance is provided only to the intended patient, the present invention electronically sends the patient identification information to the labelling unit. In the labelling unit, the labelling mechanism which can be, but is not limited to, a sticky label or other comparable method is used to display the set of patient information on the selected syringe or other comparable holding unit. When the specified volume of the specific controlled substance is extracted and available for distribution, the set of dosage information for the specific medical records is updated on the external server. More specifically, the present invention electronically updates the set of dosage information on the external server by eliminating the specified volume of the specific controlled substance from the specific medical record. Thus, duplicate orders are not processed for the specific medical record.

Although the invention has been explained in relation to its preferred embodiment, it is to be understood that many other possible modifications and variations can be made without departing from the spirit and scope of the invention as hereinafter claimed.

What is claimed is:

1. A method for monitoring and dispensing controlled substances, the method comprises the steps of:
providing a plurality of medical records managed by an external server;
providing a substance storage unit, wherein a plurality of controlled substances is stored within the storage unit;
providing a current quantity volume for each of the plurality of controlled substances;
(A) receiving a selection through a computing device, wherein the selection corresponds to a specific medical record from the plurality of medical records, wherein each of the plurality of medical records comprises at least one controlled substance data;
(B) forwarding the specific medical record to the substance storage unit;
(C) analyzing the specific medical record in order to match the at least one controlled substance data with a specific controlled substance from the plurality of controlled substances;
(D) extracting a specified volume of the specific controlled substance from the plurality of controlled substances, wherein the specified volume of the specific controlled substance is derived from the at least one controlled substance data;
(E) transferring the specified volume of the specific controlled substance from a filling station to a dispensing location of the dispensing unit, wherein the specified volume is transferred through a sample conveyance system;
(F) subtracting the specified volume from the current quantity volume of the specific controlled substance and updating the specific controlled substance with a new current quantity volume;
(G) transferring the specified volume of the specific controlled substance to a labelling unit with the dispensing unit;
(H) labelling the specified volume of the specific controlled substance with the at least one controlled substance data through a labelling mechanism;
(I) transferring the specified volume of the specific controlled substance to a distribution unit through a sample exiting system; and
(J) executing steps (A) through (I) for each of the plurality of medical records.

2. The method for monitoring and dispensing controlled substances, the method as claimed in claim 1 further comprises the steps of:
providing an authorized user account, wherein the authorized user account is managed by the external server;
providing a threshold refill level for each of the plurality of controlled substances;
continuously monitoring the new current quantity volume of the specific controlled substance through a volume measuring mechanism;
periodically comparing the new current quantity volume of the specific controlled substance with the threshold refill level of the specific controlled substance;
notifying the authorized user account through the external server,
if the threshold refill level is greater than the new current quantity volume of the specific controlled substance; and
prompting the authorized user account to refill the specific controlled substance.

3. The method for monitoring and dispensing controlled substances, the method as claimed in claim 2 further comprises the steps of:
measuring the new current quantity volume by weight through a weight sensor of the volume measuring mechanism.

4. The method for monitoring and dispensing controlled substances, the method as claimed in claim 2 further comprises the steps of:
measuring the new current quantity volume through a fluid volume detecting sensor of the volume measuring mechanism.

5. The method for monitoring and dispensing controlled substances, the method as claimed in claim 1 further comprises the steps of:
providing an authorized user account, wherein the authorized user account is managed by the external server;
electronically sending a notification to the authorized user account through the external server,
if a detection sensor is triggered with the specified volume of the specific controlled substance is in the distribution unit; and
prompting the authorized user account to remove the specified volume of the specific controlled substance from the distribution unit.

6. The method for monitoring and dispensing controlled substances, the method as claimed in claim 1 further comprises the steps of:
providing a set of patient identification information and a set of dosage information for each of the plurality of medical records;
electronically sending the patient identification information of the specific medical record to the labelling unit; and
electronically updating the set of dosage information on the external server for the specific medical record by eliminating the specified volume of the specific controlled substance from the specific medical record.

7. The method for monitoring and dispensing controlled substances, the method as claimed in claim 1, wherein each of the plurality of controlled substances is selected from a group consisting of Hydromorphone, Methadone, Meperidine, Oxycodone, Morphine, Fentanyl, and Codeine.

8. The method for monitoring and dispensing controlled substances, the method as claimed in claim 1 further comprises the steps of:
providing a plurality of pipettes for the dispensing unit; and
transferring the specified volume of the specific controlled substance to a selected pipette from the plurality of individual pipettes, wherein the selected pipette is in fluid communication with the specific controlled substance within the substance storage unit.

9. The method for monitoring and dispensing controlled substances, the method as claimed in claim 8 further comprises the steps of:
providing a plurality of syringes for the labelling unit; and
labelling a selected syringe with the at least one controlled substance data, wherein the selected syringe is operatively coupled with the selected pipette.
